(19) United States
(12) Patent Application Publication
(10) Pub. No.: US 2010/0159777 A1
(43) Pub. Date: Jun. 24, 2010

(54) Thermoplastic Starch Formed from an Enzymatically Debranched Starch

(75) Inventors: James H. Wang, Appleton, WI (US); Bo Shi, Neenah, WI (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 12/337,798

(22) Filed: Dec. 18, 2008

(51) Int. Cl. B32B 5/02; C12P 19/04; C09K 3/00

(52) U.S. Cl. 442/400; 435/101; 252/182.12; 442/327; 442/401

(57) ABSTRACT

A thermoplastic starch for use in a melt-processed composition (e.g., fiber, nonwoven web, etc.) is provided. The thermoplastic starch contains an enzymatically debranched starch and a plasticizer. By selectively controlling certain parameters of the enzymatic modification process (e.g., temperature, enzyme and starch concentrations, reaction time, isolation method, etc.), the present inventors have discovered that a native starch may be hydrolyzed in a highly efficient manner to form compositions having a comparably lower weight average molecular weight and viscosity, which are particularly suitable for use in the formation of thermoplastic starches for use in melt processing applications.

### BACKGROUND OF THE INVENTION

Thermoplastic starches, either alone or in combination with other polymers, are often used in the manufacture of articles for which water or biological degradation are considered important. The thermoplastic starch is typically formed by plasticizing a native starch with a functional plasticizer or mixture of plasticizers, such as polyfunctional alcohols (e.g., ethylene glycol, propylene glycol, or glycerol). Conventional thermoplastic starches, however, are often problematic in that they absorb moisture and age during storage, exhibit processing problems, and lack the requisite mechanical strength, ductility and toughness for many applications. Various techniques were thus developed in an attempt to improve the properties of thermoplastic starch. U.S. Pat. No. 6,933,335 to Berger, et al., for instance, describes a technique that involves extruding a mixture of a thermoplastic starch and at least one hydrophobic polymer with the addition of a hydrolysis component based on polyvinyl acetate, lower functional alcohols and/or water, and an acidic catalyst (e.g., dibutyl tin oxide). According to Berger, et al., the acidic catalyst enhances the transesterification or crosslinking of the starch, the hydrophobic polymer, and hydrolysis component. For this reason, the starch component of the blend has a molecular weight that is only minimally reduced relative to native starch.

Despite the techniques developed, it has still proven problematic to form melt-extruded compositions (e.g., fibers, nonwoven webs, etc.) from thermoplastic starches. Thermoplastic starch fibers, for example, typically require polymers of appropriate molecular weights and suitable melt viscosity for processing. It is often difficult, however, to achieve both mechanical strength and water/biological degradation from such polymers. As such, a need currently exists for a thermoplastic starch that exhibits good mechanical properties and is capable of water and/or biological degradation.

### SUMMARY OF THE INVENTION

In accordance with one embodiment of the present invention, a thermoplastic starch is disclosed that comprises from about 40 wt. % to about 98 wt. % of at least one enzymatically debranched starch and from about 2 wt. % to about 60 wt. % of at least one plasticizer. The enzymatically debranched starch has an amylose content of about 50 wt. % or more. Further, the thermoplastic starch has an apparent melt viscosity of from about 1 to about 100 Pascal-seconds, determined at a temperature of 160° C. and a shear rate of 1000 sec⁻¹.

In accordance with another embodiment of the present invention, a method for forming a thermoplastic starch is disclosed. The method comprises reacting a native starch with an enzyme within a solution, thereby forming a debranched starch having an amylose content of about 50 wt. % or more. The debranched starch is isolated from the solution. The isolated, debranched starch is melt blended with a plasticizer to form a thermoplastic starch. Debranched starches constitute from about 40 wt. % to about 98 wt. % of the thermoplastic starch and plasticizers constitute from about 2 wt. % to about 60 wt. % of the thermoplastic starch. Further, the thermoplastic starch has an apparent melt viscosity of from about 1 to about 100 Pascal-seconds, determined at a temperature of 160° C. and a shear rate of 1000 sec⁻¹.

Other features and aspects of the present invention are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a schematic illustration of a method for forming thermoplastic starch fibers in accordance with one embodiment of the present invention;

FIG. 2 is a graphical illustration of the amylose content of the samples of Example 5 (25 and 50 active units (AU) per gram starch), taken at various reaction times;

FIG. 3 is a graphical illustration of the amylose content of the samples of Example 6 (5, 10, 15, and 20 active units (AU) per gram starch), taken at various reaction times;

FIG. 4 is a graphical illustration of the amylose content of the samples of Example 7 taken at various reaction times;

FIG. 5 is a graphical illustration of the molecular weight of the samples of Example 8 (10 and 20 active units (AU) per gram starch); and

FIG. 6 is a graphical illustration of the apparent viscosity of the samples of Example 8 (native corn thermoplastic starch (“TPS”) and modified corn starch TPS) at various shear rates.

Repeat use of references characters in the present specification and drawings is intended to represent same or analogous features or elements of the invention.

### DETAILED DESCRIPTION OF REPRESENTATIVE EMBODIMENTS

### Definitions

As used herein the term “nonwoven web” generally refers to a web having a structure of individual fibers or threads which are interlaid, but not in an identifiable manner as in a knitted fabric. Examples of suitable nonwoven fabrics or webs include, but are not limited to, meltblown webs, spunbond webs, bonded carded webs, airlaid webs, coform webs, hydraulically entangled webs, and so forth.

As used herein, the term “meltblown web” generally refers to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 micrometers in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 micrometers, and are often between about 5 to about 20 micrometers.

### Detailed Description

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a thermoplastic starch for use in a melt-processed composition (e.g., fiber, nonwoven web, etc.). The thermoplastic starch contains an enzymatically debranched starch and a plasticizer. By selectively controlling certain parameters of the enzymatic modification process (e.g., temperature, enzyme and starch concentrations, reaction time, isolation method, etc.), the present inventors have discovered that a native starch may be hydrolyzed in a highly efficient manner to form compositions having a comparably lower weight average molecular weight and viscosity, which are particularly suitable for use in the formation of thermoplastic starches for use in melt processing applications. In this regard, various embodiments of the present invention will now be described in more detail.

### I. Thermoplastic Starch

A. Enzymatically Debranched Starch

Starch is a natural polymer produced in many plants, such as seeds of cereal grains, such as corn, waxy corn, wheat, sorghum, rice, and waxy rice; tubers, such as potatoes; roots, such as tapioca (i.e., cassava and manioc), sweet potato, and arrowroot; and the pith of the sago palm. Regardless of the source, starch is composed of amylose and amylopectin. Amylopectin is a branched polysaccharide in which linear chains of α-1,4 D-glucose residues are joined by α-1,6 glucosidic linkages. Amylose is a linear polysaccharide made of D-glucopyranose units linked together by α-1,4 glucosidic linkages. Amylose is essentially a linear polymer having a molecular weight in the range of 100,000-500,000 grams per mole, whereas amylopectin is a highly branched polymer having a molecular weight of up to several million.

The starch may contain different percentages of amylose and amylopectin. Low amylose native starches are particularly desirable due to their low cost, such as those having an amylose content less than about 50 wt. %, in some cases from about 5 wt. % to about 40 wt. %, and in some cases, from about 10 wt. % to about 35 wt. %. One example of such a starch is native corn starch, which typically contains about 30 wt. % amylose and about 70 wt. % amylopectin. Such low amylose starches typically have a weight average molecular weight (“M_{w}”) ranging from about 5,000,000 to about 25,000,000 grams per mole, in some embodiments from about 5,500,000 to about 15,000,000 grams per mole, and in some embodiments, from about 6,000,000 to about 12,000,000 grams per mole. It is generally difficult, however, to melt process low amylose starches through a small orifice (e.g., extruder die or spinneret) during melt extrusion because of the physical properties associated with a high content of amylopectin molecules, which are highly branched and have a relatively high molecular weight.

Thus, in accordance with the present invention, such native starches are treated with one or more enzymes that at least partially debranch the amylopectin molecules to impart a more linear orientation to the molecule and reduce its molecular weight. The debranching enzyme(s) are selected to be highly specific for certain locations of the polymer (e.g., α-1,6-glucosidic branch linkages) so that the molecule is not cut into chains that are too small for use in melt extrusion applications, as often occurs in chemical modification processes. For example, debranching enzymes that can selectively attack α-1,6-glucosidic branch linkages of amylopectin include isoamylases (E.C. 3.2.1.68) and pullulanases (E.C. 3.2.1.41, also termed α-1,6-glucosidase). Examples of suitable isoamylases may include, but not limited to, the isoamylase derived from the thermophilic acrhaebacterium Sulfolobus acidocaldarius (Maruta, K et al., (1996), Biochimica et Biophysica Acta 1291, p. 177-181), isoamylase from Rhodothermus marinus and isoamylase from Pseudomonas, such as Pseudomonas amyloderamosa (e.g. Pseudomonas amyloderamosa isoamylase, disclosed in EMBL database accession number J03871 or GeneBank accession number N90389). Likewise, examples of suitable pullulanases may include, but not limited to, the pullulanase derived from Pyrococcus or a protein engineered pullulanase from a Bacillus strain, such as Bacillus acidopullulyticus (e.g., Promozyme™), Bacillus deramificans, Bacillus amyloliquiefaciens (e.g., Multifect™, Genencor International, Inc.), or Bacillus deramificans pullulanase (disclosed in GeneBank accession number Q68699). Other suitable enzymes are described in U.S. Pat. No. 7,374,922 to Bisgard-Frantzen, et al.; U.S. Pat. No. 7,399,623 to Miller, et al.; U.S. Pat. No. 4,971,723 to Chiu; and U.S. Pat. No. 4,560,651 to Nielsen, et al., all of which are incorporated herein in their entirety by reference thereto for all purposes.

To initiate enzyme treatment, a native starch granule may be initially dispersed in an aqueous solution and heated at a sufficient temperature to effect gelatinization, which disrupts associative bonding of the starch molecules within the granule. The concentration of the native starch and enzyme in the aqueous solution may be selectively controlled to achieve a viscosity that allows the starch to be sufficiently mixed and dissolved to form a substantially homogenous solution. Native starch(es), for instance, may constitute from about 1 wt. % to about 25 wt. %, in some embodiments from about 2 wt. % to about 20 wt. %, and in some embodiments, from about 5 wt. % to about 15 wt. % of the solution. Likewise, aqueous solvent(s) (e.g., water) may constitute from about 75 wt. % to about 99 wt. %, in some embodiments from about 80 wt. % to about 98 wt. %, and in some embodiments, from about 85 wt. % to about 95 wt. % of the solution. The amount of enzyme employed depends upon the type of enzyme and its activity. Typically, however, the enzyme is employed in an amount of from about 5 to about 60 active units (“AU”), in some embodiments from about 10 to about 40 AU, and in some embodiments, from about 15 to about 30 AU per gram of starch. One (1) active unit is generally defined as the amount of an enzyme that will catalyze the formation of one (1) μmol or one (1) equivalent reducing potential of product per minute, determined under certain conditions. In the case of pullulanase, for instance, an active unit may be the amount of the enzyme that will form one (1) equivalent reducing potential of glucose per minute from Pullulan, determined at a pH of 4.5 and a temperature of 60° C.

The temperature and pH of the solution may also be adjusted to optimize the enzyme activity. For instance, the temperature of the solution is typically kept between about 55° C. and 85° C., in some cases between about 60° C. and about 80° C., and in some cases, between about 65° C. and about 75° C. The pH of the solution is likewise typically kept at a level below about 6.0, and in some embodiments, from about 4.0 to about 5.0. Buffers, such as acetates, phosphates, citrates, or the salts of other weak acids may be added to ensure that the pH will be at the optimum level. The time that the enzymatically-catalyzed reaction is permitted to continue may vary depending on a variety of factors, including the enzymatic activity level, starch concentration, pH, temperature, desired degree of branching, etc. It may be desired, for instance, to allow the reaction to proceed until essentially complete debranching has occurred. For example, the reaction may be allowed to proceed for a time of from about 10 to about 300 minutes, in some embodiments from about 15 to about 250 minutes, and in some embodiments, from about 20 to about 150 minutes.

Once formed, the debranched starch may then be isolated from the solution. The present inventors have discovered, for instance, that effective isolation of the debranched starch may be accomplished by forming a starch precipitate. Any non-solvent for the starch may be added to the solution to initiate the formation of a starch precipitate. One type of suitable non-solvent may include alcohols, such as methanol, ethanol, n-propanol, isopropanol, butanol, and so forth. Still other suitable non-solvents for the starch may include, for instance, triglycerides; ketones (e.g., acetone, methyl ethyl ketone, and methyl isobutyl ketone); esters (e.g., ethyl acetate, butyl acetate, diethylene glycol ether acetate, and methoxypropyl acetate); amides (e.g., dimethylformamide, dimethylacetamide, dimethylcaprylic/capric fatty acid amide and N-alkylpyrrolidones); nitriles (e.g., acetonitrile, propionitrile, butyronitrile and benzonitrile); sulfoxides or sulfones (e.g., dimethyl sulfoxide (DMSO) and sulfolane); and so forth. To achieve the desired degree of precipitation, the alcohol is typically employed in a relatively high amount. For example, the volume ratio of the alcohol to the resulting starch solution (e.g., water, debranched starch, etc.) may be from about 0.5:1 to about 20:1, in some embodiments from about 1:1 to about 10:1, and in some embodiments, from about 1.5:1 to about 5:1. The solution is typically allowed to stand until substantial equilibrium is achieved between the supernatant and the precipitate. The precipitate may then be isolated from the supernatant using any known techniques, such as by centrifugation, filtration, etc. The isolated debranched starch precipitate may then be washed and dried (e.g., to a low moisture content, typically 3-8%) after isolation to allow for handling and storage prior to further processing. Examples of drying techniques include spray drying, flash drying, tray drying, belt drying, and sonic drying.

The debranched starch generally contains a high amylose content, such as about 50 wt. % or more, in some embodiments from about 50 wt. % to about 95 wt. %, and in some embodiments, from about 52 wt. % to about 90 wt. %. Such high amylose content, debranched starches typically have a weight average molecular weight (“M_{w}”) of from about 250,000 to about 5,000,000 grams per mole, in some embodiments from about 500,000 to about 3,000,000 grams per mole, and in some embodiments, from about 800,000 to about 2,000,000 grams per mole. Due to its relatively low molecular weight, the high amylose content, debranched starch may be more readily used in melt extruded compositions.

B. Plasticizer

A plasticizer is also employed in the present invention to further enhance the ability of the debranched starch to be melt processed. Such plasticizers can soften the starch and cause inner starch chains to absorb water and swell. This swelling will, at some point, cause the outer shell to rupture and result in an irreversible destructurization of the starch granule. Once destructurized, the starch polymer chains, which are initially compressed within the granules, may stretch out and form a generally disordered intermingling of polymer chains. Upon resolidification, however, the chains may reorient themselves to form crystalline or amorphous solids having varying strengths depending on the orientation of the starch polymer chains. Because the starch is thus capable of melting and resolidifying at certain temperatures, it is generally considered a “thermoplastic starch.”

Suitable plasticizers may include, for instance, polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, and sorbitol), polyols (e.g., ethylene glycol, glycerol, propylene glycol, dipropylene glycol, butylene glycol, and hexane triol), etc. Also suitable are hydrogen bond forming organic compounds which do not have hydroxyl groups, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as copolymers of ethylene and acrylic acid, polyethylene grafted with maleic acid, polybutadiene-co-acrylic acid, polybutadiene-co-maleic acid, polypropylene-co-acrylic acid, polypropylene-co-maleic acid, and other hydrocarbon based acids. A low molecular weight plasticizer is preferred, such as less than about 20,000 g/mol, preferably less than about 5,000 g/mol and more preferably less than about 1,000 g/mol.

The relative amount of the debranched starch and plasticizer employed in the thermoplastic starch may vary depending on a variety of factors, such as the desired molecular weight, the type of starch, the affinity of the plasticizer for the starch, etc. Typically, however, the debranched starch constitutes from about 40 wt. % to about 98 wt. %, in some embodiments from about 50 wt. % to about 95 wt. %, and in some embodiments, from about 60 wt. % to about 90 wt. % of the thermoplastic starch. Likewise, the plasticizer typically constitutes from about 2 wt. % to about 60 wt. %, in some embodiments from about 5 wt. % to about 50 wt. %, and in some embodiments, from about 10 wt. % to about 40 wt. % of the thermoplastic starch. It should be understood that the weight of starch referenced herein includes any bound water that naturally occurs in the starch before mixing it with other components to form the thermoplastic starch. Starches, for instance, typically have a bound water content of about 5% to 16% by weight of the starch.

Of course, other additives may also be employed in the thermoplastic starch to facilitate its use in various types of compositions, such as dispersion aids, wetting agents, stabilizers, colorants, opacifiers, and so forth. Dispersion aids, for instance, may be employed to help create a uniform dispersion of the starch/plasticizer mixture and retard or prevent separation of the thermoplastic starch into constituent phases. Likewise, the dispersion aids may also improve the water dispersibility of the substrate. When employed, the dispersion aid(s) typically constitute from about 0.01 wt. % to about 10 wt. %, in some embodiments from about 0.1 wt. % to about 5 wt. %, and in some embodiments, from about 0.5 wt. % to about 4 wt. % of the thermoplastic starch.

Although any dispersion aid may generally be employed in the present invention, surfactants having a certain hydrophilic/lipophilic balance (“HLB”) may improve the long-term stability of the composition. The HLB index is well known in the art and is a scale that measures the balance between the hydrophilic and lipophilic solution tendencies of a compound. The HLB scale ranges from 1 to approximately 50, with the lower numbers representing highly lipophilic tendencies and the higher numbers representing highly hydrophilic tendencies. In some embodiments of the present invention, the HLB value of the surfactants is from about 1 to about 20, in some embodiments from about 1 to about 15 and in some embodiments, from about 2 to about 10. If desired, two or more surfactants may be employed that have HLB values either below or above the desired value, but together have an average HLB value within the desired range.

One particularly suitable class of surfactants for use in the present invention are nonionic surfactants, which typically have a hydrophobic base (e.g., long chain alkyl group or an alkylated aryl group) and a hydrophilic chain (e.g., chain containing ethoxy and/or propoxy moieties). For instance, some suitable nonionic surfactants that may be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C₈-C₁₈) acids, condensation products of ethylene oxide with long chain amines or amides, condensation products of ethylene oxide with alcohols, fatty acid esters, monoglyceride or diglycerides of long chain alcohols, and mixtures thereof. In one particular embodiment, the nonionic surfactant may be a fatty acid ester, such as a sucrose fatty acid ester, glycerol fatty acid ester, propylene glycol fatty acid ester, sorbitan fatty acid ester, pentaerythritol fatty acid ester, sorbitol fatty acid ester, and so forth. The fatty acid used to form such esters may be saturated or unsaturated, substituted or unsubstituted, and may contain from 6 to 22 carbon atoms, in some embodiments from 8 to 18 carbon atoms, and in some embodiments, from 12 to 14 carbon atoms. In one particular embodiment, mono- and di-glycerides of fatty acids may be employed in the present invention.

### II. Melt Processing

The thermoplastic starch of the present invention may be formed by melt blending the components together in an extruder. The mechanical shear and heat provided by the extruder and allows the components to be blended together in a highly efficient manner. Batch and/or continuous melt blending techniques may be employed in the present invention. For example, a mixer/kneader, Banbury mixer, Farrel continuous mixer, single-screw extruder, twin-screw extruder, roll mill, etc., may be utilized. One particularly suitable melt-blending device is a co-rotating, twin-screw extruder (e.g., USALAB twin-screw extruder available from Thermo Electron Corporation of Stone, England or an extruder available from Werner-Pfreiderer from Ramsey, N.J.). Such extruders may include feeding and venting ports and provide high intensity distributive and dispersive mixing. For example, a starch may be initially fed to a feeding port of the twin-screw extruder. Thereafter, a plasticizer may be injected into the starch. Alternatively, the components may be simultaneously fed to the feed throat of the extruder or separately at a different point along its length.

Regardless, the materials are blended under high shear/pressure and heat to ensure sufficient mixing. For example, melt blending typically occurs at a temperature of from about 50° C. to about 200° C., in some embodiments, from about 60° C. to about 180° C., and in some embodiments, from about 70° C. to about 150° C.

Likewise, the apparent shear rate during melt blending may range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

As a result of melt blending with the plasticizer, the weight average molecular weight and viscosity of the resulting thermoplastic starch may be substantially reduced in comparison to the initial debranched starch, thereby rending it more suitable for many applications. For example, the thermoplastic starch may have a weight average molecular weight ranging from about 50,000 to about 1,000,000 grams per mole, in some embodiments from about 75,000 to about 800,000 grams per mole, and in some embodiments, from about 100,000 to about 600,000 grams per mole. The thermoplastic starch may also have a relatively low apparent melt viscosity, such as from about 1 to about 100 Pascal seconds (Pa·s), in some embodiments from about 5 to about 60 Pa·s, and in some embodiments, from about 20 to about 50 Pa·s, as determined at a temperature of 160° C. and a shear rate of 1000 sec⁻¹. The melt flow index (190° C., 2.16 kg) of the thermoplastic starch may also range from about 0.05 to about 50 grams per 10 minutes, in some embodiments from about 0.1 to about 15 grams per 10 minutes, and in some embodiments, from about 0.5 to about 5 grams per 10 minutes.

Ill. Melt-Processed Compositions

The thermoplastic starch of the present invention may be incorporated into any known melt-processed composition, such as a fiber, nonwoven web (e.g., spunbond web, meltblown web, and so forth), etc. The composition may contain a single layer or multiple layers and may also contain additional materials such that it is considered a composite. Regardless, in certain embodiments, the thermoplastic starch may constitute at least about 50 wt. %, in some embodiments from about 60 wt. % to about 99 wt. %, and in some embodiments, from about 75 to about 95 wt. % of the polymer content of the composition.

Various known techniques may be employed to form a melt-processed composition. Fibers, for instance, may be formed from the thermoplastic starch. Such fibers may have any desired configuration, including monocomponent, multicomponent (e.g., sheath-core configuration, side-by-side configuration, segmented pie configuration, island-in-the-sea configuration, and so forth), and/or multiconstituent (e.g., polymer blend). Any of a variety of processes may be used to form fibers in accordance with the present invention. For example, the thermoplastic starch may be extruded through a spinneret, quenched, and drawn into the vertical passage of a fiber draw unit. The fibers may then be cut to form staple fibers having an average fiber length in the range of from about 3 to about 80 millimeters, in some embodiments from about 4 to about 65 millimeters, and in some embodiments, from about 5 to about 50 millimeters. If desired, the staple fibers may then be incorporated into a nonwoven web as is known in the art, such as bonded carded webs, through-air bonded webs, etc.

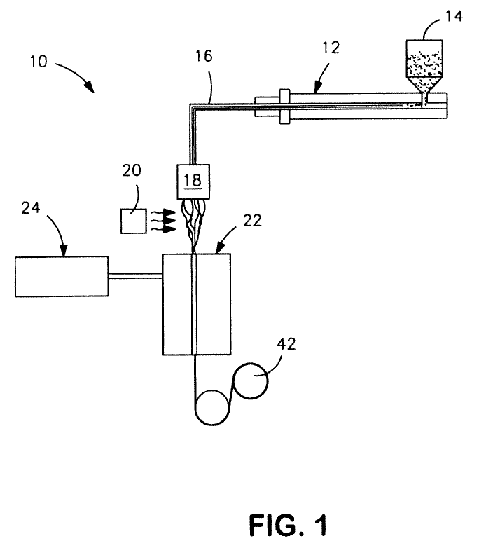

The fibers may also be deposited onto a foraminous surface to form a nonwoven web. Referring to FIG. 1, for example, one embodiment of a method for forming spunbond fibers is shown. In FIG. 1, for instance, the raw materials (e.g., debranched starch and plasticizer) are fed into an extruder 12 from a hopper 14. The raw materials may be provided to the hopper 14 using any conventional technique and in any state. The extruder 12 is driven by a motor (not shown) and heated to a temperature sufficient to extrude the melted polymer. Once formed, the thermoplastic starch may be subsequently fed to another extruder in a fiber formation line. Alternatively, as shown in FIG. 1, the thermoplastic starch may be directly formed into a fiber through a polymer conduit 16 to a spinneret 18. Spinnerets for extruding filaments are well known to those of skill in the art. For example, the spinneret 18 may include a housing containing a spin pack having a plurality of plates stacked one on top of each other and having a pattern of openings arranged to create flow paths for directing polymer components. The spinneret 18 also has openings arranged in one or more rows. The openings form a downwardly extruding curtain of filaments when the polymers are extruded therethrough. The process 10 also employs a quench blower 20 positioned adjacent the curtain of filaments extending from the spinneret 18. Air from the quench air blower 20 quenches the filaments extending from the spinneret 18. The quench air may be directed from one side of the filament curtain as shown in FIG. 1 or both sides of the filament curtain. A fiber draw unit or aspirator 22 is positioned below the spinneret 18 and receives the quenched filaments. Fiber draw units or aspirators for use in melt spinning polymers are well-known in the art. Suitable fiber draw units for use in the process of the present invention include a linear fiber aspirator of the type shown in U.S. Pat. Nos. 3,802,817 and 3,423,255, which are incorporated herein in their entirety by reference thereto for all relevant purposes. The fiber draw unit 22 generally includes an elongate vertical passage through which the filaments are drawn by aspirating air entering from the sides of the passage and flowing downwardly through the passage. A heater or blower 24 supplies aspirating air to the fiber draw unit 22. The aspirating air draws the filaments and ambient air through the fiber draw unit 22. Thereafter, the filaments are formed into a coherent web structure by randomly depositing the filaments onto a forming surface 26 (optionally with the aid of a vacuum) and then bonding the resulting web using any known technique.

After quenching, the filaments are drawn into the vertical passage of the fiber draw unit 22 by a flow of a gas such as air, from the heater or blower 24 through the fiber draw unit. The flow of gas causes the filaments to draw or attenuate which increases the molecular orientation or crystallinity of the polymers forming the filaments. The filaments are deposited through the outlet opening of the fiber draw unit 22 and onto a godet roll 42. If desired, the fibers collected on the godet roll 42 may optionally be subjected to additional in line processing and/or converting steps (not shown) as will be understood by those skilled in the art. For example, staple fibers may be formed by “cold drawing” the collected fibers at a temperature below their softening temperature to the desired diameter, and thereafter crimping, texturizing, and/or and cutting the fibers to the desired fiber length.

If desired, the fibers may also be directly formed into a coherent web structure by randomly depositing the fibers onto a forming surface (optionally with the aid of a vacuum) and then bonding the resulting web using any known technique. For example, an endless foraminous forming surface may be positioned below the fiber draw unit and receive the filaments from an outlet opening. A vacuum may be positioned below the forming surface to draw the filaments and consolidate the unbonded nonwoven web. Once formed, the nonwoven web may then be bonded using any conventional technique, such as with an adhesive or autogenously (e.g., fusion and/or self-adhesion of the fibers without an applied external adhesive). Autogenous bonding, for instance, may be achieved through contact of the fibers while they are semi-molten or tacky, or simply by blending a tackifying resin and/or solvent with the polymer(s) used to form the fibers. Suitable autogenous bonding techniques may include ultrasonic bonding, thermal bonding, through-air bonding, calendar bonding, and so forth. For example, the web may be further bonded or embossed with a pattern by a thermo-mechanical process in which the web is passed between a heated smooth anvil roll and a heated pattern roll. The pattern roll may have any raised pattern which provides the desired web properties or appearance. Desirably, the pattern roll defines a raised pattern which defines a plurality of bond locations which define a bond area between about 2% and 30% of the total area of the roll. Exemplary bond patterns include, for instance, those described in U.S. Pat. No. 3,855,046 to Hansen et al., U.S. Pat. No. 5,620,779 to Levy et al., U.S. Pat. No. 5,962,112 to Haynes et al., U.S. Pat. No. 6,093,665 to Sayovitz et al., as well as U.S. Design Pat. No. 428,267 to Romano et al.; U.S. Pat. No. 390,708 to Brown; U.S. Pat. No. 418,305 to Zander et al.; U.S. Pat. No. 384,508 to Zander, et al.; U.S. Pat. No. 384,819 to Zander, et al.; U.S. Pat. No. 358,035 to Zander, et al.; and U.S. Pat. No. 315,990 to Blenke, et al., all of which are incorporated herein in their entirety by reference thereto for all purposes. The pressure between the rolls may be from about 5 to about 2000 pounds per lineal inch. The pressure between the rolls and the temperature of the rolls is balanced to obtain desired web properties or appearance while maintaining cloth like properties. As is well known to those skilled in the art, the temperature and pressure required may vary depending upon many factors including but not limited to, pattern bond area, polymer properties, fiber properties and nonwoven properties.

In addition to spunbond webs, a variety of other nonwoven webs may also be formed from the thermoplastic starch in accordance with the present invention, such as meltblown webs, bonded carded webs, wet-laid webs, airlaid webs, coform webs, hydraulically entangled webs, etc. For example, the thermoplastic starch may be extruded through a plurality of fine die capillaries into a converging high velocity gas (e.g., air) streams that attenuate the fibers to reduce their diameter. Thereafter, the meltblown fibers are carried by the high velocity gas stream and are deposited on a collecting surface to form a web of randomly dispersed meltblown fibers. Alternatively, the polymer may be formed into a carded web by placing bales of fibers formed from the thermoplastic starch into a picker that separates the fibers. Next, the fibers are sent through a combing or carding unit that further breaks apart and aligns the fibers in the machine direction so as to form a machine direction-oriented fibrous nonwoven web. Once formed, the nonwoven web is typically stabilized by one or more known bonding techniques.

### IV. Articles

The melt-processed thermoplastic starch composition of the present invention may be used in a wide variety of applications. For example, the composition may be in the form of individual fibers or a nonwoven web incorporated into a “medical product”, such as gowns, surgical drapes, facemasks, head coverings, surgical caps, shoe coverings, sterilization wraps, warming blankets, heating pads, and so forth. The fibers or web may also be used in an “absorbent article” that is capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, mitt wipe, and so forth; medical absorbent articles, such as garments, fenestration materials, underpads, bedpads, bandages, absorbent drapes, and medical wipes; food service wipers; clothing articles; pouches, and so forth. Materials and processes suitable for forming such articles are well known to those skilled in the art. Absorbent articles, for instance, typically include a substantially liquid-impermeable layer (e.g., outer cover), a liquid-permeable layer (e.g., bodyside liner, surge layer, etc.), and an absorbent core. In one embodiment, for example, a nonwoven web formed according to the present invention may be used to form an outer cover of an absorbent article. If desired, the nonwoven web may be laminated to a liquid-impermeable film that is either vapor-permeable or vapor-impermeable.

The present invention may be better understood with reference to the following examples.

### Test Methods

Amylose Content

Amylose content was determined by a spectroscopic method using Hewlett Packard 8453 UV-Vis Spectrometer. The path length was 1 centimeter, the wavelength was selected to be 600 nanometers, and the cuvette was quartz. The reference media was deionized water.

Standard solutions were initially prepared by weighing 0.0, 0.01, 0.03, and 0.05 grams of a reference standard obtained from Sigma-Aldrich of St. Louis, Mo. (100 wt. % amylose) into separate 25 mL volumetric flasks and 15 mL aliquots of dimethyl sulfoxide (DMSO) were added. The flasks were placed in an 85° C. water bath for around 15 minutes. The flasks were then taken out of the water bath, allowed to cool, and diluted to mark with deionized water. One (1) milliliter aliquot was then placed into separate 50 mL volumetric flasks, and approximately 30 mL of deionized water was added followed by a 5 mL addition of IKI solution, which was then diluted to mark with deionized water.

Sample solutions were also prepared by weighing approximately 0.050 grams of the sample into a 25 mL volumetric flask and adding 15 milliliters of DMSO. The flask was placed in an 85° C. water bath for around 15 minutes, allowed to cool, and diluted to mark with deionzied water. One (1) milliliter aliquot was then placed into a 50 mL volumetric flask and approximately 30 mL of deionized water was added followed by a 5 mL addition of IKI solution, which was then diluted to mark with deionized water.

A calibration curve was developed by plotting the measured absorbency readings of the standard solutions using an HP 8453 UV-Vis Spectrometer versus the amylose content of each standard. To determine the amylose content in a sample, absorbency readings were taken and compared to the calibration curve to find the amylose content corresponding to the absorbency readings.

Apparent Melt Viscosity:

The rheological properties of polymer samples were determined using a Göttfert Rheograph 2003 capillary rheometer with WinRHEO version 2.31 analysis software. The setup included a 2000-bar pressure transducer and a 30/1:0/180 roundhole capillary die. Sample loading was done by alternating between sample addition and packing with a ramrod. A 2-minute melt time preceded each test to allow the polymer to completely melt at a test temperature (160° C.). The capillary rheometer determined the apparent melt viscosity (Pa·s) at various shear rates, such as 100, 200, 500, 1000, 2000, and 4000 s⁻¹. The resultant rheology curve of apparent shear rate versus apparent melt viscosity gave an indication of how the polymer would run at that temperature in an extrusion process.

### EXAMPLE 1

Native corn starch (obtained from Cargill Inc. of Minneapolis, Minn.) was debranched using a pullulanase enzyme. The pullulanase was Multifect® 1000 U/mL (lot#1680679631) and was acquired from Genencor International (Rochester, N.Y.). A round bottom glass vessel from Wilmad-LabGlass (Buena, N.J.) was initially filled with 500 milliliters of tap water and placed in a heating mantle. A RW20DZM stirring apparatus from IKA Labortechnik (Staufen, Germany) was used to agitate the suspension. An Optichem® temperature probe and heat controller from Chemglass (Vineland, N.J.) was connected to a heating pad from Glass Col® (Terre Haute, Ind.) to maintain the desired temperature throughout the experiment. The temperature probe was placed in the water and the heating knob was set to 68° C. (resulted in a water temperature of 70° C.). While heating and stirring, the native corn starch was slowly added to achieve a 10 wt. % solution. The mixture was continuously stirred at approximately 68° C. for a total of 1 hour. The temperature was then reset to 60° C. in order to cool the suspension. While cooling, 2.05 grams of sodium acetate and 1.5 milliliters of acetic acid (˜99.7% purity, Sigma-Aldrich of St. Louis, Mo.) were added to adjust the pH to a level of 4.7.

When the temperature was close to 60° C., 2.5 milliliters of the enzyme was added to the starch suspension. This dosage, including enzyme dosages in other experiments, was estimated. More specifically, the manufacture of the enzyme (Genencor) indicated that a minimum of 1000 active units was present per milliliter of enzyme solution. Thus, if enzyme activity unit of 50 per gram of starch was desired, 50 grams of starch is selected for the experiment and the amount of pullulanase needed was calculated as 50 activity unit/gm×50 gm of starch/1000 activity unit=2.5 mL. The enzyme was allowed to react for 100 minutes. Upon completion, the stirrer and heater were turned off and the reaction vessel was removed. The starch solution was then combined with twice its volume of isopropyl alcohol (purity of 90-100%, obtained from Mallinckrodt Baker, Inc. of Paris, Ky.), causing a precipitate to form. The solution of 1500 mL was filtered with filter paper (#3 or #4) from Whatman (Maidstone, Ky.) using a vacuum to speed up filtration. The remaining starch was transferred to paper to air dry overnight. A blender (or mortar & pestle) was used to crush the dried polymer into fine powder.

### EXAMPLE 2

A starch/pullulanase solution was formed as substantially described in Example 1, except that 140 grams of native starch and 700 grams of water were employed to result in a 20 wt. % solution in relation to amount water. The reaction time was 1 hour. In this particular example, however, the propeller used to stir the solution was too small and the starch formed large globs of polymer that did not dissolve. In the second experiment, a 10 wt. % solution was prepared and a larger propeller was used, resulting in a homogenous starch suspension. Therefore, the 10 wt. % solution was used in the rest of the examples.

### EXAMPLE 3

A starch/pullulanase solution was formed as substantially described in Example 1, except that a water temperature of 90° C. was employed rather than 70° C. The reaction time was 1 hour. In this particular example, the higher temperature caused the start to over-gelatinize, which made it difficult to stir the solution.

### EXAMPLE 4

A starch/pullulanase solution was formed as substantially described in Example 1, except that isopropyl alcohol was not used to precipitate the starch. The reaction time was 1 hour. At the end of the experiment, the solution in a shallow pan was simply dried in an oven at 130° C. overnight. In this particular example, oven-drying resulted in a brittle starch slab that was unsuitable for amylose titration and molecular weight determination by gel permeation chromatography.

### EXAMPLE 5

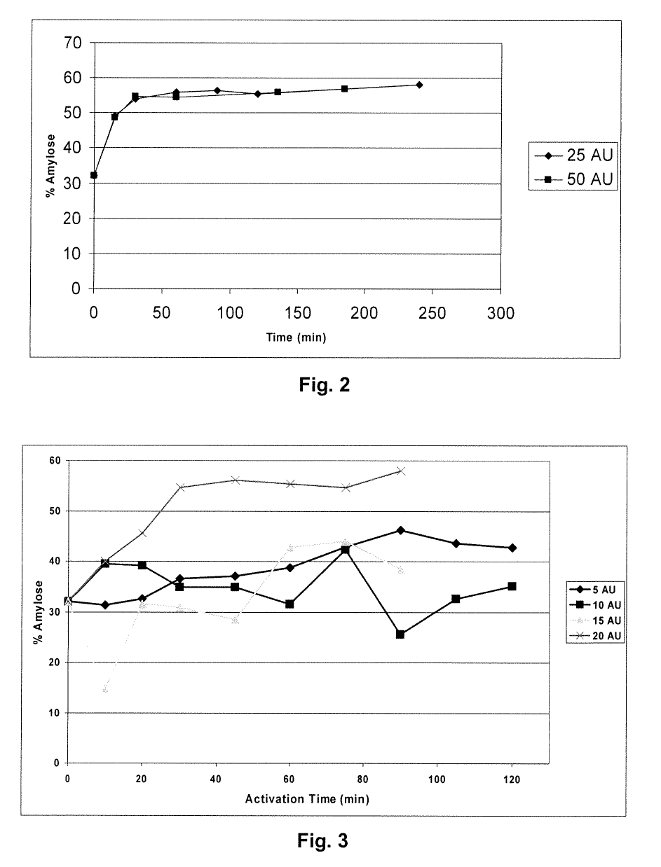

The effect of enzyme reaction times on the amylose content of the debranched starch was tested. More specifically, debranched starch precipitates were formed as substantially described in Example 1. A single batch was made with 25 active units (AU) enzyme per gram starch. Samples were taken at time intervals of 15, 30, 60, 90, 120, and 240 minutes and filtered by a Whatman® No. 4 filtration paper. The samples filtered at the time intervals of 15, 30, and 90 minutes each had a volume of about 10 milliliters. The samples filtered at the time intervals of 60 and 120 minutes each had a volume of about 40 milliliters. Finally, the sample filtered at the time interval of 240 minutes had a volume of about 380 milliliters. A similar process was also followed for another batch using 50 AU enzyme per gram starch. The results are shown in FIG. 2. As shown, laboratory analysis indicated that the amylose content leveled off relatively early, i.e., at approximately one (1) hour. Also, 25 active units performed as well as 50 active units.

### EXAMPLE 6

The effect of enzyme concentration on the amylose content of the debranched starch was tested. More specifically, debranched starch precipitates were formed as substantially described in Example 1 with enzymes at a concentration of 5, 10, 15, and 20 active units (AU) enzyme per gram starch. The reaction time was fixed at 1 hour. The results are shown in FIG. 3. As shown, the amylose content fluctuated at low levels of enzyme. At 20 AU, the amylose level tended to smooth out.

### EXAMPLE 7

The ability to debranch native corn starch (obtained from Cargill Inc. of Minneapolis, Minn.) using an isoamylase enzyme was demonstrated. The isoamylase was acquired from Sigma-Aldrich (St. Louis, Mo.) as (15284-1MU, lot#047K1452 and was dissolved into a 1 liter solution of 0.8M [(NH₄)₂SO₄], with an estimated concentration of 1 million activity units per liter. However, the definition for activity unit by Sigma-Aldrich was different from the one by Genencor International (See Example 1) by a factor of 160. Thus, the isoamylase from Sigma-Aldrich had an equivalent activity unit of 6250 per liter in order to be comparable to enzyme activity unit used by Genencor International. In addition, optimal conditions for isoamylase were at 40° C. and a pH of 3.5. Therefore, a buffer solution was prepared using a mixture of 70.2 mL of 0.1M citric acid solution and 29.8 mL of 0.2M potassium phosphate solution.

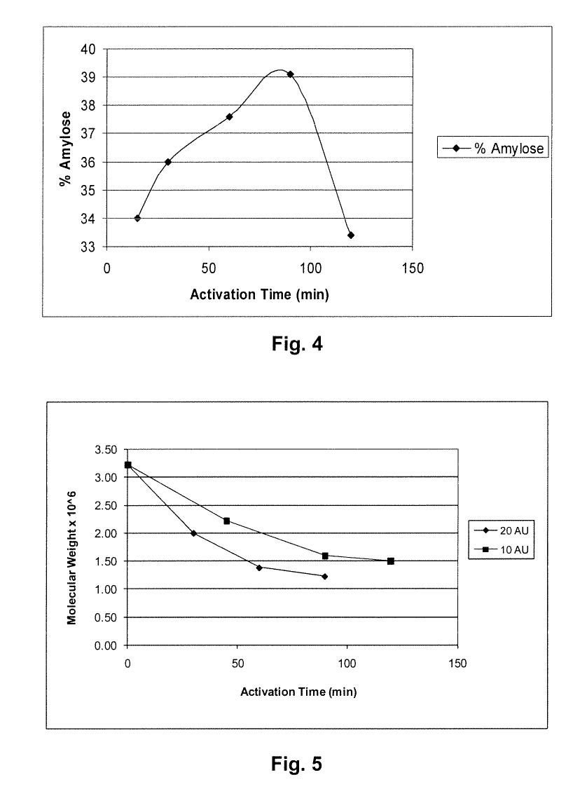

A starch/isoamylase solution was formed as substantially described in Example 1, except that 50 grams of native starch and 200 grams of water and 100 mL of buffer solution were employed initially. After starch gelatinization, a 200 mL of the isoamylase stock solution was taken and added, resulting in isoamylose activity unit at 25 per gram of starch, which was at 10 wt. % of solution with respect to a total amount of liquids in the reaction vessel. The reaction time was 2 hours. The modified starches, sampled at different times during the two hour experiment, were analyzed for amylose content. The results are shown in FIG. 4. As illustrated, amylose content increased initially from 34% for the sample collected after 10 minutes of enzymatic starch modification to 39% for the sample collected after 100 minutes of starch modification by isomaylase. The last data point for amylose in the modified starch was collected at 120 minutes and was unexpectedly lower than the previous sample.

Isoamylose activity unit at 50 per gram of starch was also carried out at similar conditions above using 50 grams of native corn starch, 200 mL tap water, 100 mL buffer solution, and 400 mL isoamylase stock solution. The final starch to a total amount liquid in the reaction vessel was 7.1% (slightly less than 10%), although enzyme activity units remained at 50 per gram of starch. After the isomaylase was added, the aliquot samples were collected at 15, 30, 60, 90, 120, and 150 minutes for filtration, drying, and amylose determinations. The results indicated that amylose content in all six samples was ranged from 25-27.5%, which is at level of native corn starch without any modification by enzyme.

### EXAMPLE 8

The effect of enzyme concentration and reaction time on the molecular weight of the debranched starch was tested. More specifically, debranched starch precipitates were formed as substantially described in Example 1 with enzymes at a concentration of 10 and 20 activity units (AU) enzyme per gram starch and at reactions times ranging from 30 to 120 minutes. Samples of the resulting debranched starches were sent to the American Polymer Standards Corporation (Mentor, Ohio) for a determination of weight average molecular weight. The results are shown in FIG. 5. As indicated, the weight molecular weight consistently decreased as the enzyme reacted with the starch, both with 20 AU and 10 AU enzyme concentrations. This decrease confirmed that the enzyme is in fact cutting the amylopectin molecules into smaller pieces.

### EXAMPLE 9

The ability to form a thermoplastic starch in accordance with the present invention was demonstrated. Initially, about ten (10) batches of debranched corn starch (each having a weight of 75 grams) were made as substantially described in Example 1, except that the enzyme concentration was 25 active units per gram starch and the reaction time was 1 hour.

2 wt. % Excel P-40S (mono-diglyceride, Kao Corp. of Japan) was then dry mixed with the debranched corn starch in a kitchen mixer and added to a K-Tron gravimetric feeder (Model KCL-QX4, K-Tron North America, Pitman, N.J.) that fed the material into a Thermo Prism™ USALAB 16 twin screw extruder (Thermo Electron Corp., Stone, England). The extruder had 11 zones, numbered consecutively 0-10 from the feed hopper to the die. The temperature profile of zones 1 to 10 of the extruder was 112° C., 120° C., 130° C., 140° C., 140° C., 140° C., 130° C., 115° C., 90° C., and 80° C., respectively. The screw speed was set at 150 rpm to achieve a torque of between 55˜60% and a pressure of 1.9 to 2.0 MM Pa during processing. The starch feeding rate was fixed at 1.5 pounds per hour. The starch was fed to the feed throat of the extruder (un-heated, before zone 1 of the extruder). Glycerin (Emery 916 Glycerine 99.7% CP/USP, Cognis Corporation of Cincinnati, Ohio) was injected into zone 1 of the extruder using a plasticizer gear pump (Bodine Electric Company, Grand Island, N.Y.) to achieve a plasticizer concentration of 30 wt. %. In some cases, a vent was opened to release steam generated. The resulting strand cooled down through a cooling belt (Minarik Electric Company, Glendale, Calif.). A pelletizer (Emerson Industrial Controls, Grand Island, N.Y.) was used to cut the strand to produce thermoplastic starch (“TPS”) pellets. The appearance of the pellets was light in color, with small white flecks.

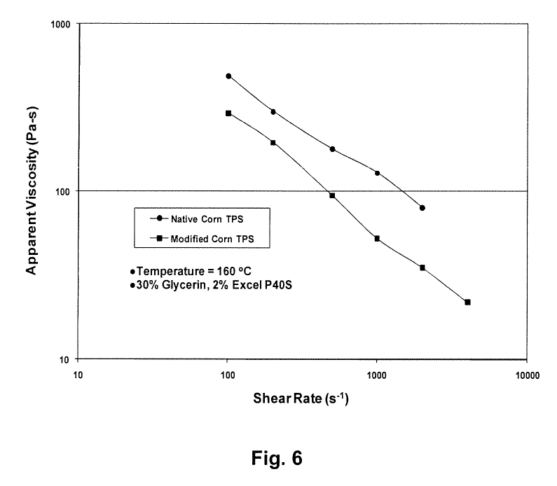

The melt rheology testing of the resulting thermoplastic starch pellets was then determined and compared to thermoplastic starch pellets formed from unmodified native corn starch. The results are shown in FIG. 6. As indicated, the pellets formed from debranched starch had a lower viscosity than those formed from native corn starch. At 1000 s⁻¹, for example, when the native corn TPS had an apparent viscosity of approximately 125 Pa-s, the debranched corn TPS had an apparent viscosity of approximately 40 Pa-s, 68% lower than the native corn TPS viscosity. Such a dramatic decrease indicated that the debranched corn TPS would have a higher melt flow rate, which is desirable for certain applications, such as injection molding, fiber spinning, etc.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A thermoplastic starch comprising from about 40 wt. % to about 98 wt. % of at least one enzymatically debranched starch and from about 2 wt. % to about 60 wt. % of at least one plasticizer, wherein the enzymatically debranched starch has an amylose content of about 50 wt. % or more, and further wherein the thermoplastic starch has an apparent melt viscosity of from about 1 to about 100 Pascal-seconds, determined at a temperature of 160° C. and a shear rate of 1000 sec⁻¹.

2. The thermoplastic starch composition of claim 1, wherein the enzymatically debranched starch is derived from corn, wheat, sorghum, rice, potatoes, tapioca, sweet potato, arrowroot; the pith of the sago palm, or a combination thereof.

3. The thermoplastic starch composition of claim 1, wherein the enzymatically debranched starch is derived from corn.

4. The thermoplastic starch composition of claim 1, wherein the enzymatically debranched starch has an amylose content of from about 52 wt. % to about 90 wt. %.

5. The thermoplastic starch composition of claim 1, wherein the enzymatically debranched starch has a weight average molecular weight of from about 250,000 to about 5,000,000 grams per mole.

6. The thermoplastic starch composition of claim 1, wherein the enzymatically debranched starch has a weight average molecular weight of from about 800,000 to about 2,000,000 grams per mole.

7. The thermoplastic starch composition of claim 1, wherein the enzymatically debranched starch constitutes 60 wt. % to about 90 wt. % of the thermoplastic starch and the plasticizer constitutes from about 10 wt. % to about 40 wt. % of the thermoplastic starch.

8. The thermoplastic starch composition of claim 1, wherein the thermoplastic starch has an apparent melt viscosity of from about 5 to about 60 Pascal-seconds, determined at a temperature of 160° C. and a shear rate of 1000 sec⁻¹.

9. The thermoplastic starch composition of claim 1, wherein the plasticizer includes a polyol.

10. The thermoplastic starch composition of claim 1, wherein the thermoplastic starch is melt-extruded into the form of a fiber.

11. A nonwoven web comprising a plurality of fibers, wherein the fibers contain a thermoplastic starch formed from about 40 wt. % to about 98 wt. % of at least one enzymatically debranched starch and from about 2 wt. % to about 60 wt. % of at least one plasticizer, wherein the enzymatically debranched starch has an amylose content of about 50 wt. % or more, and further wherein the thermoplastic starch has an apparent melt viscosity of from about 1 to about 100 Pascal-seconds, determined at a temperature of 160° C. and a shear rate of 1000 sec⁻¹.

12. The nonwoven web of claim 11, wherein the nonwoven web includes a spunbond web, meltblown web, or a combination thereof.

13. A method for forming a thermoplastic starch, the method comprising:
reacting a native starch with an enzyme within a solution, thereby forming a debranched starch having an amylose content of about 50 wt. % or more;
isolating the debranched starch from the solution; and
melt blending the isolated, debranched starch with a plasticizer to form a thermoplastic starch, wherein debranched starches constitute from about 40 wt. % to about 98 wt. % of the thermoplastic starch and plasticizers constitute from about 2 wt. % to about 60 wt. % of the thermoplastic starch, further wherein the thermoplastic starch has an apparent melt viscosity of from about 1 to about 100 Pascal-seconds, determined at a temperature of 160° C. and a shear rate of 1000 sec⁻¹.

14. The method of claim 13, wherein the starch is derived from corn, wheat, sorghum, rice, potatoes, tapioca, sweet potato, arrowroot; the pith of the sago palm, or a combination thereof.

15. The method of claim 13, wherein the native starch has an amylose content of less than about 50 wt. %.

16. The method of claim 13, wherein the debranched starch has an amylose content of from about 52 wt. % to about 90 wt. %.

17. The method of claim 13, wherein debranched starches constitute from about 60 wt. % to about 90 wt. % of the thermoplastic starch and plasticizers constitute from about 10 wt. % to about 40 wt. % of the thermoplastic starch.

18. The method of claim 13, wherein the thermoplastic starch has an apparent melt viscosity of from about 5 to about 60 Pascal-seconds, determined at a temperature of 160° C. and a shear rate of 1000 sec⁻¹.

19. The method of claim 13, wherein the plasticizer includes a polyol.

20. The method of claim 13, wherein the enzyme includes a pullulanase, isoamylase, or a combination thereof.

21. The method of claim 13, wherein the solution is at a temperature of from about 65° C. to about 75° C.

22. The method of claim 13, wherein the pH of the solution is from about 4.0 to about 5.0.

23. The method of claim 13, wherein the enzyme and the native starch are reacted for a period of time of from about 20 to about 150 minutes.

24. The method of claim 13, wherein native starches constitute from about 2 wt. % to about 20 wt. % of the solution.

25. The method of claim 13, wherein the enzyme is employed in an amount of from about 5 to about 60 active units per gram of starch, determined at a pH of 4.5 and a temperature of 60° C.

26. The method of claim 13, wherein the enzyme is employed in an amount of from about 10 to about 40 active units per gram of starch, determined at a pH of 4.5 and a temperature of 60° C.

27. The method of claim 13, wherein the debranched starch is isolated by precipitating the debranched starch to form a precipitate, and separating the precipitate from the solution.

28. The method of claim 27, wherein the precipitate is formed by contacting the solution with a non-solvent of the starch.

29. The method of claim 28, wherein the non-solvent includes an alcohol.

30. The method of claim 29, wherein the alcohol includes isopropyl alcohol.

31. The method of claim 28, wherein the volume ratio of the non-solvent to the solution is from about 1:1 to about 10:1.

32. The method of claim 28, wherein the volume ratio of the non-solvent to the solution is from about 1.5:1 to about 5:1.

33. The method of claim 13, wherein melt blending occurs at a temperature of from about 50° C. to about 200° C.

34. The method of claim 13, further comprising extruding the thermoplastic starch onto a surface.
